(19) United States
(12) Patent Application Publication
(10) Pub. No.: US 2021/0270823 A1
(43) Pub. Date: Sep. 2, 2021

(54) DIAGNOSTIC AGENT, DIAGNOSTIC KIT, AND DIAGNOSTIC METHOD

(71) Applicant: CANON KABUSHIKI KAISHA, Tokyo (JP)

(72) Inventors: Masaru Sugita, Tokyo (JP); Masataka Yashima, Tokyo (JP); Kyohei Wada, Tokyo (JP); Koichi Sato, Kanagawa (JP); Akira Tsuboyama, Tokyo (JP)

(21) Appl. No.: 17/178,695

(22) Filed: Feb. 18, 2021

(30) Foreign Application Priority Data
Feb. 28, 2020 (JP) 2020-033560

(51) Int. Cl. G01N 33/545; G01N 33/58; G01N 21/64

(52) U.S. Cl. CPC G01N 33/545; G01N 2021/6439; G01N 21/6428; G01N 33/582

(57) ABSTRACT

A diagnostic agent for use in detecting a target substance in a specimen, including a mixture of: a photosensitizer-loaded particle containing a photosensitizer that generates singlet oxygen during light irradiation; a fluorescent dye-loaded particle containing a fluorescent dye that changes in luminescent characteristics depending on an oxidation reaction with the singlet oxygen; and an aggregation inhibitor that inhibits aggregation of the photosensitizer-loaded particle and the fluorescent dye-loaded particle, characterized in that at least one of the photosensitizer-loaded particle and the fluorescent dye-loaded particle has the property of binding to the target substance, and the surface of the photosensitizer-loaded particles and the surface of the fluorescent dye-loaded particles have a stimulus-responsive polymer, a diagnostic kit for producing the diagnostic agent, and a diagnostic method with the use of a component of the diagnostic agent.

### BACKGROUND OF THE INVENTION

### Field of the Invention

The present invention relates to a diagnostic agent, a diagnostic kit, and a diagnostic method for use in detecting a target substance.

### Description of the Related Art

Conventionally, quantitatively detecting substances in the fields of chemistry, biomedicine, and environment has been required, and above all, latex agglutination has been used as a method for detecting a target substance with low concentration.

The latex agglutination is a method of detecting or quantifying a target substance contained in a sample by evaluating the degree of aggregation of latex particles in the sample. The latex particles carry a substance that specifically binds to the target substance, thereby allowing the target substance to be detected.

In recent years, detecting smaller amounts of target substances has been required in each technical field. In the latex agglutination, however, the latexes are less likely to be cross-linked with each other by binding between the target substance and a specifically binding substance supported on the latex, as the amount of the target substance is smaller. For that reason, insufficient aggregation may be achieved for detection.

In addition, the latex particles may non-specifically bind to various non-target substance trace substances contained in the sample to be subjected to the detection, inducing aggregation. For that reason, depending on the type of the sample to be subjected to the detection, there is a need to take measures to avoid non-specific binding between the latex particles and the non-target substances in order to increase the detection accuracy.

Thus, in fields such as biomedicine, the method of using an enzyme-substrate reaction has been also widely employed. Examples of the method of using the enzyme-substrate reaction include the Enzyme-Linked Immuno Sorbent Assay (ELISA) method and the Chemiluminescent Enzyme Immunoassay (CLEIA) method. In these methods, for example, a substance that specifically binds to a target substance is first bound to the target substance. Subsequently, an enzyme is bound to the substance that specifically binds to the target substance. In this regard, the enzyme carries another substance that further specifically binds to the substance that specifically binds to the target substance. Thus, the enzyme can be specifically bound to the substance that specifically binds to the target substance. Thereafter, the substrate for the enzyme is added, and the degree of the reaction catalyzed by the enzyme is measured, thereby detecting the target substance. In the method of using the enzyme-substrate reaction, the detection sensitivity can be increased by using, as the substrate, a luminescent substance such as a fluorescent dye, and it may be possible to detect the target substance in small amounts. The method of using the enzyme-substrate reaction, however, typically requires a washing step, and the method is not considered excellent in terms of rapidity and simplicity of operation.

Japanese Patent Application Laid-Open No. 2009-162532 proposes a method of using magnetic particles that aggregate in response to a stimulus as a simple method for detecting a small amount of target substance without requiring any washing step.

### SUMMARY OF THE INVENTION

Since the method of using magnetic particles as described in Japanese Patent Application Laid-Open No. 2009-162532 requires the operation of the particles by a magnetic field, the use of the device may be complicated for control of the external magnetic field. In addition, the production of the magnetic particles themselves may take a lot of work.

The present invention has been made in view of the above-mentioned problems, and an object of the present invention is to provide a diagnostic agent, a diagnostic kit, and a diagnostic method, which enable quick, inexpensive, simple, highly sensitive, and highly accurate detection or quantification of a target substance.

A diagnostic agent according to an aspect of the present invention is a diagnostic agent for use in detecting a target substance in a specimen, including a mixture of: a photosensitizer-loaded particle containing a photosensitizer that generates singlet oxygen during light irradiation; a fluorescent dye-loaded particle containing a fluorescent dye that changes in luminescent characteristics depending on an oxidation reaction with the singlet oxygen; and an aggregation inhibitor that inhibits aggregation of the photosensitizer-loaded particle and the fluorescent dye-loaded particle, characterized in that at least any one of the photosensitizer-loaded particle and the fluorescent dye-loaded particle has the property of binding to the target substance, and the surface of the photosensitizer-loaded particles and the surface of the fluorescent dye-loaded particles have a stimulus-responsive polymer.

A diagnostic agent according to an another aspect of the present invention is a diagnostic agent for use in detecting a target substance in a specimen, including: a photosensitizer-loaded particle containing a photosensitizer that generates singlet oxygen by light irradiation; a fluorescent dye-loaded particle containing a fluorescent dye that changes in luminescent characteristics depending on an oxidation reaction with the singlet oxygen; an aggregation inhibitor that has the property of inhibiting aggregation of the photosensitizer-loaded particle and the fluorescent dye-loaded particle by binding to the target substance; and an aggregation enhancer that accelerates aggregation of the photosensitizer-loaded particle and the fluorescent dye-loaded particle, characterized in that at least any one of the photosensitizer-loaded particle and the fluorescent dye-loaded particle has the property of binding to the target substance.

Furthermore, a diagnostic kit according to another aspect of the present invention is a diagnostic kit for use in producing the diagnostic agent mentioned above, including the photosensitizer-loaded particle, the fluorescent dye-loaded particle, and the aggregation inhibitor, characterized in that at least any one of the photosensitizer-loaded particle, the fluorescent dye-loaded particle, and the aggregation inhibitor is stored separately.

Furthermore, a diagnostic method according to still another aspect of the present invention is a diagnostic method for detecting a target substance in a specimen, characterized in that the diagnostic method includes: a step of obtaining a test mixture by mixing the specimen, a photosensitizer-loaded particle containing a photosensitizer that generates singlet oxygen during light irradiation, a fluorescent dye-loaded particle containing a fluorescent dye that changes in luminescent characteristics depending on an oxidation reaction with the singlet oxygen, and an aggregation inhibitor that inhibits aggregation of the photosensitizer-loaded particle and the fluorescent dye-loaded particle, where at least any one of the photosensitizer-loaded particle and the fluorescent dye-loaded particle has the property of binding to the target substance, and the surface of the photosensitizer-loaded particles and the surface of the fluorescent dye-loaded particles have a stimulus-responsive polymer; a step of changing an environmental condition for the test mixture to change the property of the stimulus-responsive polymer; a step of irradiating the test mixture with first excitation light; and a step of irradiating the test mixture with second excitation light and detecting light emitted from the test mixture.

Further features of the present invention will become apparent from the following description of exemplary embodiments.

### DESCRIPTION OF THE EMBODIMENTS

A diagnostic agent according to the present invention is a diagnostic agent for use in detecting a target substance in a specimen, including a mixture of: a photosensitizer-loaded particle containing a photosensitizer that generates singlet oxygen during light irradiation; a fluorescent dye-loaded particle containing a fluorescent dye that changes in luminescent characteristics depending on an oxidation reaction with the singlet oxygen; and an aggregation inhibitor that inhibits aggregation of the photosensitizer-loaded particle and the fluorescent dye-loaded particle, characterized in that at least one of the photosensitizer-loaded particle and the fluorescent dye-loaded particle has the property of binding to the target substance, and the surface of the photosensitizer-loaded particles and the surface of the fluorescent dye-loaded particles have a stimulus-responsive polymer.

Another diagnostic agent according to the present invention is a diagnostic agent for use in detecting a target substance in a specimen, including a photosensitizer-loaded particle containing a photosensitizer that generates singlet oxygen by light irradiation; a fluorescent dye-loaded particle containing a fluorescent dye that changes in luminescent characteristics depending on an oxidation reaction with the singlet oxygen; an aggregation inhibitor that has the property of inhibiting aggregation of the photosensitizer-loaded particle and the fluorescent dye-loaded particle by binding to the target substance; and an aggregation enhancer that accelerates aggregation of the photosensitizer-loaded particle and the fluorescent dye-loaded particle, characterized in that at least any one of the photosensitizer-loaded particle and the fluorescent dye-loaded particle has the property of binding to the target substance.

In the present invention, the stimulus-responsive polymer present on the surface of the photosensitizer-loaded particle and the surface of the fluorescent dye-loaded particle changes in property in response to the stimulus, thereby causing the photosensitizer-loaded particle and the fluorescent dye-loaded particle to aggregate or bind to each other. More specifically, the property of the stimulus-responsive polymer changes in response to the stimulus, thereby allowing the photosensitizer-loaded particle and the fluorescent dye-loaded particles to present in close proximity to each other. The both particles in close proximity allows the singlet oxygen generated by the photosensitizer-loaded particle during light irradiation to reach the fluorescent dye contained in the fluorescent dye-loaded particle, and the fluorescent dye changes in luminescent characteristics depending on the oxidation reaction. This change in luminescent characteristics can be used as a detection signal.

In addition, at least any one of the photosensitizer-loaded particle and the fluorescent dye-loaded particle has the property of binding to a target substance. More specifically, the surface of at least any one of the photosensitizer-loaded particle and the fluorescent dye-loaded particle has a site that binds to the target substance (hereinafter, also referred to as a target binding site). In order to detect the target substance with high accuracy, the target binding site preferably specifically binds to the target substance.

The target binding site of the surface of the photosensitizer-loaded particle or fluorescent dye-loaded particle binds to the target substance, thereby changing the dispersibility of the particles. Thus, when aggregation of the photosensitizer-loaded particle and fluorescent dye-loaded particle is induced by applying a stimulus to the sample, the aggregation of the particles with the target substance bound thereto is inhibited. More specifically, in the case where the sample contains little or no target substance, the photosensitizer-loaded particle and the fluorescent dye-loaded particle mostly aggregate in response to the stimulus, thus increasing the change in luminescent characteristics in the case of light irradiation between before and after applying the stimulus. In contrast, in the case where the sample contains the target substance in large amount, the target binding site of the surface of the photosensitizer-loaded particle or the fluorescent dye-loaded particle bind to the target substance, thereby inhibiting the aggregation of the particles even in the case of applying a stimulus, and then reducing the change in luminescent characteristics in the case of light irradiation between before and after applying the stimulus. Based on the foregoing principle, it is possible to detect or quantify the target substance in the sample quickly, inexpensively, and simply with high sensitivity and high accuracy, without using a washing step.

In the present invention, there is no limitation on the polymer compound mainly constituting the photosensitizer-loaded particle and the fluorescent dye-loaded particle, and polymer compounds can be used, which are typically used for forming particles for use in methods such as latex agglutination.

The photosensitizer-loaded particle containing a photosensitizer that generates singlet oxygen during light irradiation may be an inorganic particle, an organic particle, or a mixed particle thereof. The average particle size of the photosensitizer-loaded particle is preferably 5 nm or more and 100 μm or less. As the target substance, low-molecular-weight or high-molecular-weight chemical substances are conceivable, such as sugars, proteins, lipids, and complex substances thereof, and cells and biological tissues. From the foregoing, the average particle size of the photosensitizer-loaded particle is preferably 20 nm or more and 10 μm or less, more preferably 80 nm or more and 1 μm or less. In addition, the diffusion phenomenon allows the singlet oxygen generated from the sensitizer in the photosensitizer-loaded particle to reach the fluorescent dye in the fluorescent dye-loaded particle at sufficient concentration without being deactivated, thereby efficiently causing the change in fluorescence. Thus, the average particle size of the photosensitizer-loaded particle is particularly preferably 80 nm or more and 500 nm or less.

The photosensitizer-loaded particle is preferably a spherical particle that has a uniform particle size as uniform as possible, and the main structure is thus preferably a particle of an organic polymer compound. As the organic polymer compound, a polymer compound such as polystyrene, polyethylene, polypropylene, polyacrylate, polymethacrylate, polybutadiene, polyisoprene, or polychloroprene, or a copolymer of the polymer compounds is preferably used. Further, the photosensitizer-loaded particle may contain therein a filler such as an inorganic substance. Conventionally known methods such as emulsification, suspension, emulsion polymerization, suspension polymerization are used as a method for granulating the photosensitizer-loaded particle, but the method is not to be considered particularly limited.

The aspect in which a photosensitizer is contained in the photosensitizer-loaded particle is not particularly limited, and may be any aspect as long as the photosensitizer is not dispersed and/or eluted from the photosensitizer-loaded particle when the photosensitizer-loaded particle is dispersed in the sample. Specifically, examples of the aspect include chemically binding or adsorbing a photosensitizer to a polymer compound that serves as the main structure of the photosensitizer-loaded particle by covalent bonding, hydrophobic bonding, ionic bonding, hydrogen bonding, an intermolecular force, or the like.

The photosensitizer that generates singlet oxygen during light irradiation refers to a dye that absorbs light to turn into an excited state that can convert triplet oxygen into singlet oxygen (see, e.g., Synthetic Organic Chemistry, Vol. 26, No. 3, p. 217). As the photosensitizer, an inorganic dye, an organic dye, or an organic-inorganic composite dye containing the both elements can be used. For example, the photosensitizer is at least one selected from the group consisting of a porphyrin that may have a substituent, a phthalocyanine that may have a substituent, a fluorescein that may have a substituent, and a methylene blue, and the porphyrin and the phthalocyanine may each have a central metal. In this regard, the substituents that may be included in the porphyrin, the phthalocyanine, and the fluorescein are each a halogen atom, a linear or branched alkyl group, an alkoxy group, an aryl group that may have a substituent, or a heterocyclic group that may have a substituent. The substituent that may be included in the aryl group is a halogen atom, a linear or branched alkyl group, an alkoxy group, an aryl group, or a heterocyclic group. In addition, the substituent that may be included in the heterocyclic group is a halogen atom, a linear or branched alkyl group, an alkoxy group, or an aryl group. The heterocyclic groups that may be included in the porphyrin, the phthalocyanine, and the fluorescein, and the heterocyclic group that may be included in the aryl group are each independently any group selected from the group consisting of a pyridyl group, an oxazolyl group, an oxadiazolyl group, a thiazolyl group, a thiadiazolyl group, a carbazolyl group, an acridinyl group, and a phenanthrolyl group. The number of carbon atoms in the linear or branched alkyl group mentioned above is preferably 1 or more and 20 or less, more preferably 1 or more and 5 or less.

Specific examples of the photosensitizer include compounds represented by the following formulas (1) to (5).

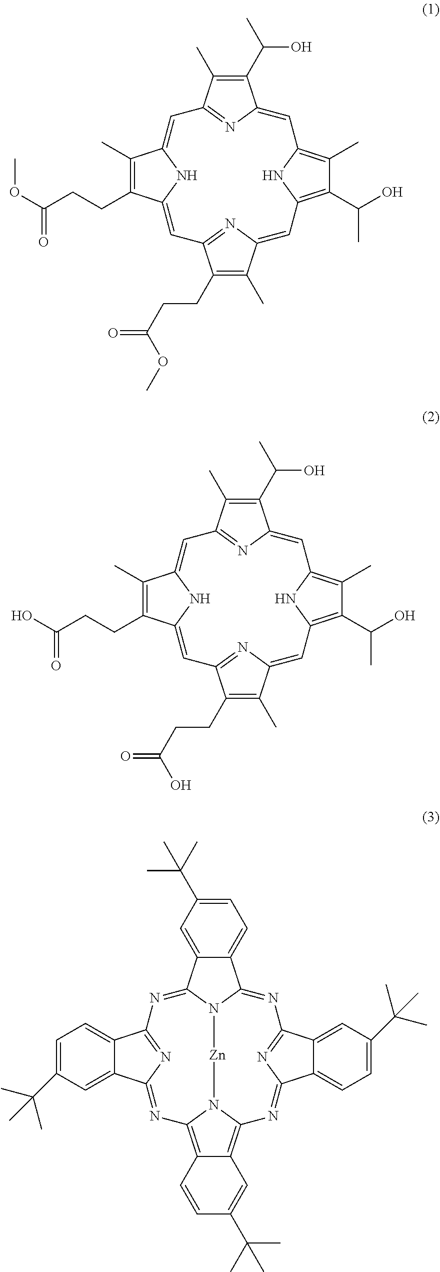

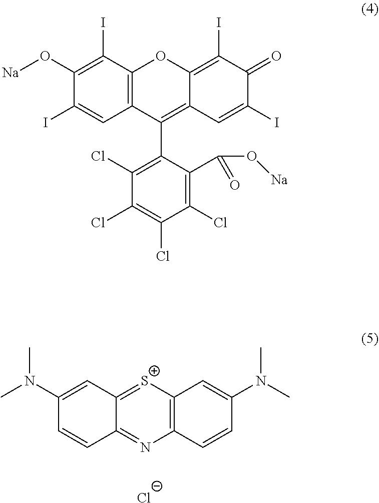

The fluorescent dye-loaded particle containing the fluorescent dye that changes in luminescence characteristics depending on the oxidation reaction with singlet oxygen may be an inorganic particle, an organic particle, or a mixed particle thereof. The average particle size of the fluorescent dye-loaded particle is preferably 5 nm or more and 100 μm or less. From the viewpoint of interaction with the conceivable target substance described above, the average particle size of the fluorescent dye-loaded particles is more preferably 20 nm or more and 10 μm or less, further preferably 80 nm or more and 1 μm or less. Further, from the viewpoint of causing singlet oxygen to reach the fluorescent dye without being deactivated at a sufficient concentration, the average particle size of the fluorescent dye-loaded particles is particularly preferably 80 nm or more and 500 nm or less.

The fluorescent dye-loaded particle is preferably a spherical particle that has a particle size as uniform as possible, and thus preferably a particle with an organic polymer compound as a binder. As the organic polymer compound, a polymer compound such as polystyrene, polyethylene, polypropylene, polyacrylate, polymethacrylate, polybutadiene, polyisoprene, or polychloroprene, or a copolymer of the polymer compounds is preferably used. Further, the fluorescent dye-loaded particle may contain therein a filler such as an inorganic substance. Conventionally known methods such as emulsification, suspension, emulsion polymerization, suspension polymerization are used as a method for granulating the fluorescent dye-loaded particle, but the method is not to be considered particularly limited.

The aspect in which a fluorescent dye is contained in the fluorescent dye-loaded particle is not particularly limited, and may be any aspect as long as the photosensitizer is not dispersed and/or eluted from the photosensitizer-loaded particle when the photosensitizer-loaded particle is dispersed in the sample. Specifically, examples of the aspect include chemically binding or adsorbing a photosensitizer to a polymer compound that serves as the main structure of the photosensitizer-loaded particle by covalent bonding, hydrophobic bonding, ionic bonding, hydrogen bonding, an intermolecular force, or the like.

As for the fluorescent dye contained in the fluorescent dye-loaded particle, “changing in luminescent characteristics” depending on the oxidation reaction with singlet oxygen specifically refers to the fact that the fluorescence wavelength of the fluorescent dye is shifted, the fluorescence disappears, or the fluorescence is produced. In addition, in the present invention, “fluorescence” is used as a term also including “phosphorescence”. As the fluorescent dye, an inorganic dye, an organic dye, or an organic-inorganic composite dye containing the both elements can be used. For example, the fluorescent dye is anthracene, that may have a substituent or benzofuran that may have a substituent. In this regard, the substituents that may be included in the anthracene and the benzofuran are each a halogen atom, a linear or branched alkyl group, an alkoxy group, an aryl group that may have a substituent, or a heterocyclic group that may have a substituent. The substituent that may be included in the aryl group is a halogen atom, a linear or branched alkyl group, an alkoxy group, an aryl group, or a heterocyclic group. In addition, the substituent that may be included in the heterocyclic group is a halogen atom, a linear or branched alkyl group, an alkoxy group, or an aryl group. The heterocyclic groups that may be included in the anthracene and the benzofuran, and the heterocyclic group that may be included in the aryl group are each independently any group selected from the group consisting of a pyridyl group, an oxazolyl group, an oxadiazolyl group, a thiazolyl group, a thiadiazolyl group, a carbazolyl group, an acridinyl group, and a phenanthrolyl group. The number of carbon atoms in the linear or branched alkyl group mentioned above is preferably 1 or more and 20 or less, more preferably 1 or more and 5 or less.

Specific examples of the fluorescent dye include compounds represented by the following formulas (6) and (7).

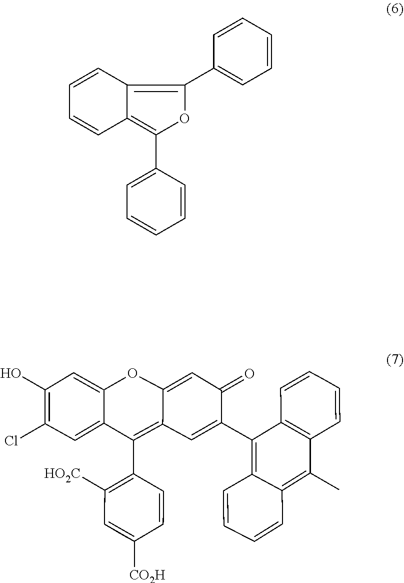

The stimulus-responsive polymer of the surface of the photosensitizer-loaded particle and the surface of the fluorescent dye-loaded particle is preferably bound to the particle surfaces in a grafted shape, that is, preferably bound to the site present on the particle surface of the polymer compound mainly constituting each particle in a grafted shape. In addition, since the target substance is typically included in water or an aqueous solvent, the stimulus-responsive polymer is preferably hydrophilic, which is bound in a grafted shape to each of the surfaces of the photosensitizer-loaded particle and fluorescent dye-loaded particle dispersed in the sample including the target substance.

Examples of the stimulus-responsive polymer include a temperature-responsive polymer and a pH-responsive polymer. Further, examples of the temperature-responsive polymer include a polymer that has a lower critical solution temperature and a polymer that has an upper critical solution temperature.

Examples of the polymer having a lower critical solution temperature include polymers obtained from N-substituted (meth)acrylamide derivatives such as N-n-propylacrylamide, N-isopropylacrylamide, N-ethylacrylamide, N,N-dimethylacrylamide, N-acryloylpyrrolidine, N-acryloylpiperidine, N-acryloylmorpholine, N-n-propylmethacrylamide, N-isopropylmethacrylamide, N-ethylmethacrylamide, N,N-dimethylmethacrylamide, N-t-butylacrylamide, N-methacryloylpyrrolidine, N-methacryloylpiperidine, and N-methacryloylmorpholine; polyoxyethylene alkylamine derivatives such as hydroxypropyl celluloses, partial acetates of polyvinyl alcohols, polyvinyl methyl ethers, (polyoxyethylene-polyoxypropylene) block copolymers, and polyoxyethylene laurylamines; polyoxyethylene sorbitan ester derivatives such as polyoxyethylene sorbitan laurates; (polyoxyethylene alkylphenyl ether) (meth)acrylates such as (polyoxyethylene nonylphenyl ether) acrylates and (polyoxyethylene octylphenyl ether) methacrylate; and polyoxyethylene (meth)acrylic acid ester derivatives such as (polyoxyethylene alkyl ether) (meth)acrylates such as (polyoxyethylene lauryl ether) acrylates and (polyoxyethylene oleyl ether) methacrylates. Furthermore, copolymers obtained from these polymers and at least two monomers for the polymers are also available. Furthermore, these polymers and copolymers may be copolymerized with other copolymerizable monomers to the extent of having a lower critical solution temperature. In the present invention, above all, a polymer obtained from at least one monomer selected from the group consisting of N-n-propylacrylamide, N-isopropylacrylamide, N-ethylacrylamide, N,N-dimethylacrylamide, N-acrylloylpyrrolidine, N-acryloylpiperidine, N-acryloylmorpholine, N-n-propylmethacrylamide, N-isopropylmethacrylamide, N-ethylmethacrylamide, N,N-dimethylmethacrylamide, N-methacryloylpyrrolidine, N-methacryloylpiperidine, and N-methacryloylmorpholine, or a copolymer of N-isopropylacrylamide and N-t-butylacrylamide is preferably available.

As an example of the polymer having an upper critical solution temperature, polymers obtained from at least one monomer selected from the group consisting of acryloyl glycinamide, acryloyl nipecotamide, acryloyl asparagine amide, acryloyl glutamine amide, and the like are available. Alternatively, the polymer may be copolymers obtained from at least two of the foregoing monomers. These polymers or copolymers may be copolymerized with other copolymerizable monomers to the extent of having an upper critical solution temperature. Examples of the copolymerizable monomers include acrylamide, acetylacrylamide, biotinol acrylate, N-biotinyl-N′-methacryloyl trimethyleneamide, acryloyl sarcosine amide, methacryl sarcosine amide, and acryloyl methyl uracil.

These polymer having a lower critical solution temperature and polymer with an upper critical solution temperature change in lyophilicity, lyophobicity, hydrophilicity, or hydrophobicity in response to the stimulus of changing the temperature, thereby causing the photosensitizer-loaded particle and the fluorescent dye-loaded particle to aggregate or bind to each other.

Examples of the pH-responsive polymer include polymers containing a group such as carboxyl, a phosphoric acid, sulfonyl, or amino as a functional group. More specifically, examples thereof include polymers including, as a copolymerizable component, an acrylic acid, a methacrylic acid, a maleic acid, a vinyl sulfonic acid, a vinyl benzene sulfonic acid, a phosphoryl ethyl (meth)acrylate, an aminoethyl methacrylate, an aminopropyl (meth)acrylamide, a dimethylaminopropyl (meth)acrylamide, or a salt thereof. These pH-responsive polymers cause the photosensitizer-loaded particle and the fluorescent dye-loaded particle to aggregate or bind in response to the stimulus of changing the pH.

In the present invention, the stimulus-responsive polymer is preferably at least any one selected from the group consisting of poly(N-alkylacrylamide), poly(N-vinylalkylamide), and polyvinyl alkyl ether. Furthermore, the stimulus-responsive polymer is more preferably poly(N-isopropylacrylamide).

The mixture included in the diagnostic agent according to the present invention contains an aggregation inhibitor that inhibits the aggregation of the photosensitizer-loaded particle and fluorescent dye-loaded particle. The aggregation inhibitor as well as the surface of the photosensitizer-loaded particle or the surface of the fluorescent dye-loaded particle, preferably has a target binding site. This makes it possible to inhibit the aggregation of the photosensitizer-loaded particle and fluorescent dye-loaded particle more effectively in the case where the sample includes a target substance. The binding between the target binding site of the aggregation inhibitor and the target substance is also preferably specific.

In the present invention, a material that accelerates agglutination may be added in inducing the aggregation of the photosensitizer-loaded particle and fluorescent dye-loaded particle. Specific examples of the material that accelerates the aggregation can include polymer compound-type aggregation enhancers such as polyacrylamide and polyallylamine.

The target binding site of the surface of the photosensitizer-loaded particle, the surface of the fluorescent dye-loaded particle, or the aggregation inhibitor can be arbitrarily selected depending on the target substance in question. The target binding site may be bound or adsorbed to the target substance by, for example, covalent bonding, ionic bonding, hydrogen bonding, an intermolecular force, or the like. In addition, in the case of employing, for example, biomolecules, sugars, proteins, lipids, and complex substances thereof, and cells and biological tissues as target substances, nucleic acids such as antibodies and DNA aptamers, glycoproteins, proteins such as protein A and protein G, glycolipids, lipoproteins, and the like can be used as the target binding site.

There is no particular restriction on the relation between the stimulus-responsive polymer and the target binding site, but a substance that is used for the target binding site is preferably present near the moiety where the stimulus-responsive polymer is lyophilic or hydrophilic and the stimulus-responsive polymer is solvated. For example, in the case where the stimulus-responsive polymer of the respective surfaces of the photosensitizer-loaded particle and fluorescent dye-loaded particle is N-isopropylacrylamide, the NH and CO of the amide structure are hydrated. In contrast, a carboxy group is introduced into the polymer compound mainly constituting the photosensitizer-loaded particle or the fluorescent dye-loaded particle, and an amino group or the like of a substance that is used for a target binding site is reacted with the carboxy group for immobilization on the particle. This allows the target binding site to be present near the amide structure in N-isopropylacrylamide, that is, the solvated moiety.

In particular, the substance that is used for the target binding site is preferably bound to, of the two ends of the stimulus-responsive polymer, the end that is not bound to the polymer compound mainly constituting the photosensitizer-loaded particle or the fluorescent dye-loaded particle, or a moiety that is close to the end. In this case, the target binding site at the end of the stimulus-responsive polymer actively in Brownian motion in the solution is high in binding reaction activity with the target substance. Thus, the target substance dispersed in the solvent and the target binding site are allowed to quickly bound to each other. In conventional latex agglutination, latex particles often have the target binding site immediately thereon. The aspect in which the photosensitizer-loaded particle or the fluorescent dye-loaded particle has the target binding site with the stimulus-responsive polymer interposed therebetween in the present invention has a clear advantage as compared with the conventional case.

Furthermore, since the stimulus-responsive polymer has lyophilicity or hydrophilicity, there is also the advantage that contaminants that interfere with the binding between the target substance and the target binding site, which can be present in the specimen, can be prevented from adsorbing or binding to the particles in the binding between the target substance and the target binding site.

Various target substances are conceivable for the target substance described above, and the target substance is not to be considered limited in any way in the present invention, but in many cases, most of the conceivable substances are present in water or an aqueous solvent. In reality, environmental measurements in rivers and the seas and medical diagnoses are conceivable. In such cases, the aggregation inhibitor preferably has a hydrophilic site and has high solubility and high dispersibility in water or an aqueous solvent. The hydrophilic site of the aggregation inhibitor preferably has a structure represented by removing one hydrogen atom from at least any one selected from the group consisting of polyethylene glycol, polyvinylpyrrolidone, polyvinyl alcohol, sodium polyacrylate, polyethyleneimine, and copolymers thereof except for one hydrogen atom.

A diagnostic kit according to the present invention is a diagnostic kit for use in producing the diagnostic agent described above, including the photosensitizer-loaded particle, the fluorescent dye-loaded particle, and the aggregation inhibitor, characterized in that at least any one of the photosensitizer-loaded particle, the fluorescent dye-loaded particle, and the aggregation inhibitor is stored separately.

In the diagnostic kit according to the present invention, the photosensitizer-loaded particle, the fluorescent dye-loaded particle, and the aggregation inhibitor are preferably all stored separately from each other. This eliminates the possibility that the photosensitizer-loaded particle, the fluorescent dye-loaded particle, and the aggregation inhibitor interact with each other before the diagnostic kit is subjected to a specimen test and then affect the test.

Next, a diagnostic method according to the present invention will be described. The diagnostic method according to the present invention is a diagnostic method for detecting a target substance in a specimen, characterized in that the diagnostic method includes: a step of obtaining a test mixture by mixing the specimen, a photosensitizer-loaded particle containing a photosensitizer that generates singlet oxygen during light irradiation, a fluorescent dye-loaded particle containing a fluorescent dye that changes in luminescent characteristics depending on an oxidation reaction with the singlet oxygen, and an aggregation inhibitor that inhibits aggregation of the photosensitizer-loaded particle and the fluorescent dye-loaded particle, where at least any one of the photosensitizer-loaded particle and the fluorescent dye-loaded particle has the property of binding to the target substance, and the surface of the photosensitizer-loaded particles and the surface of the fluorescent dye-loaded particles have a stimulus-responsive polymer; a step of changing an environmental condition for the test mixture to change the property of the stimulus-responsive polymer; a step of irradiating the test mixture with first excitation light; and a step of irradiating the test mixture with second excitation light and detecting light emitted from the test mixture. The stimulus-responsive polymer is preferably a temperature-responsive polymer.

In the diagnostic method according to the present invention, first, the specimen, the photosensitizer-loaded particle, the fluorescent dye-loaded particle, and the aggregation inhibitor are mixed to obtain a test mixture. A stimulus is applied to the obtained test mixture, thereby changing the environmental conditions for the test mixture and inducing aggregation caused by a change in the property of the stimulus-responsive polymer. Subsequently, the test mixture is irradiated with the first excitation light to generate singlet oxygen from the photosensitizer contained in the photosensitizer-loaded particle. Thus, in the case where the photosensitizer-loaded particle and the fluorescent dye-loaded particle are brought close to each other by the aggregation, the fluorescent characteristics of the fluorescent dye contained in the fluorescent dye-loaded particle are changed by the oxidation reaction with singlet oxygen. Thereafter, the test mixture is further irradiated with the second excitation light to induce fluorescence from the fluorescent dye.

At least any one of the photosensitizer-loaded particle and the fluorescent dye-loaded particle has the property of binding to the target substance. More specifically, at least any one of the photosensitizer-loaded particle and the fluorescent dye-loaded particle has a target binding site. The target binding site may be included in either the photosensitizer-loaded particle or the fluorescent dye-loaded particle, or may be included in both of the particles, and for example, for employing the amount of fluorescence derived from the fluorescent dye as a quantification measure, the fluorescent dye-loaded particle only may have a target binding site.

In the present invention, the optimum test conditions can be set by arbitrarily setting the amounts of the photosensitizer-loaded particle, the fluorescent dye-loaded particle, and the aggregation enhancer depending on the type of the target substance. For example, in the case where a minute amount of substance is used as the target substance, the amount of the photosensitizer-loaded particle may be made larger than that of the fluorescent dye-loaded particle such that the photosensitizer-loaded particle has a target binding site. Alternatively, in the case where a substance that is large in abundance is used as the target substance, the amount of the fluorescent dye-loaded particle may be made smaller than that of the photosensitizer-loaded particle such that the fluorescent dye-loaded particle has a target binding site. In this case, the test can be performed without significantly changing the amount of photosensitizer-loaded particle.

The order of mixing the specimen with the photosensitizer-loaded particle, the fluorescent dye-loaded particle, or the aggregation enhancer is not particularly restricted, and can be arbitrarily determined depending on the type of the target substance, the substance that is used for the target binding site, or the like.

In the case where the target substance decreases the dispersibility of the photosensitizer-loaded particle and fluorescent dye-loaded particle, the aggregation inhibitor preferably has a target binding site. This makes it possible to inhibit the aggregation of the photosensitizer-loaded particle or fluorescent dye-loaded particle with the target substance bound thereto.

Currently, in various fields, the quantification of substances on the pM concentration scale has been required, but the conventional methods have the problem of difficulty in achieving the quantification. The diagnostic method according to the present invention is capable of achieving the quantification with the use of the photosensitizer-loaded particle and the fluorescent dye-loaded particle on the order of several pM to 100 pM with respect to the target substance on the pM concentration scale. In this case, the photosensitizer and the fluorescent dye can be each contained in the range of approximately 0.01% by mass to 80% by mass in the photosensitizer-loaded particle and the fluorescent dye-loaded particle.

Assume that the fluorescent dye-loaded particle contains 10% by mass of a dye, the molecular weight of the dye is typically 300, the particle size of the fluorescent dye-loaded particle is 300 nm, the specific gravity of the fluorescent dye-loaded particle is 1, and the concentration of the fluorescent dye-loaded particle in the sample is 10 pM. In this case, the concentration of the dye in the sample is 28 μM, which is found to be a concentration at which fluorescence can be easily detected with the use of an excitation light device generally available in a relatively inexpensive manner. More specifically, in the present invention, one feature of the present invention is that particles that are equivalent in concentration to the target substance are used in the test, while in the detection, the use of a dye that is abundantly present in the particles, and further of fluorescence easily detected. This makes it possible to detect a small amount of target substance with high sensitivity. For example, the above-described diagnostic agent, diagnostic kit, and diagnostic method can be used in an automatic analyzer that analyzes components such as blood and urine. Furthermore, the present invention also has the advantage of requiring no magnetic-field control in a conventional method with magnetic particles used, and the advantage of being capable of suppressing the disturbance of detected signals by non-specific adsorption of contaminants that can be present in the specimen.

### EXAMPLES

[Synthesis of Particle with Stimulus-Responsive Polymer Grafted]

A latex particle dispersion was obtained by dispersing 18.5 parts by mass of latex rubber particles (trade name: LX111A2, produced by Zeon Corporation, particle size: 300 nm) (10 parts by mass of particle component) in 82 parts by mass of water.

Next, the following materials were prepared.
Ethylenediaminetetraacetic acid (EDTA): 0.0019 parts by mass
FeSO₄: 0.0005 parts by mass
HOCH₂SOONa: 0.096 parts by mass
N-isopropylacrylamide: 7 parts by mass
Mono-2-methacryloyl ethyl ester succinate: 0.07 parts by mass
Cumene hydroperoxide: 0.024 parts by mass

These materials were dissolved in a mixed solution of ethyl acetate and isopropanol. It is to be noted that the HOCH₂SOONa was added to the mixed solution under a nitrogen stream. The obtained solution was delivered by drops over 2 hours into the latex particle dispersion prepared as mentioned above. After further continuing the reaction for 2 hours, the reaction solution was put into excess acetone, centrifuged, dispersed again in acetone, and then centrifuged again to obtain particles subjected to grafting. The obtained particles were analyzed with an infrared absorption spectrum to confirm that 12.4% by mass of poly(N-isopropylacrylamide) was grafted to the latex rubber particles.

[Preparation of Photosensitizer-Loaded Particles]

A photosensitizer solution was obtained by dissolving 0.4 parts by mass of zinc tetra-tert-butyl phthalocyanine in a mixed solution of ethyl acetate and acetone. Subsequently, a photosensitizer solution was added to and then stirred in the dispersion obtained by dispersing 8 parts by mass of the above-obtained particles subjected to the grafting in water. The mixture was left for 1 hour under a nitrogen stream, and the solvent was distilled away to prepare photosensitizer-loaded particles containing a photosensitizer.

[Preparation of Fluorescent Dye-Loaded Particle]

Diphenylisobenzofuran was used in place of the zinc tetra-tert-butyl phthalocyanine in the preparation of the photosensitizer-loaded particles. In the same manner as in the preparation of the photosensitizer-loaded particles except for the foregoing use, fluorescent dye-loaded particles containing a fluorescent dye were prepared.

[Evaluation of Aggregation Inhibitor]

In the evaluation of the aggregation inhibitor, the evaluation was made with magnetic particles including avidin as a “target substance” and biotinylated polyethylene glycol as a “substance that traps the target substance”.

Magnetic particles with avidin as a target binding site (trade name: Therma-Max (registered trademark) LA avidin, produced by Wako Pure Chemical Industries, Ltd.) were suspended in water to adjust the concentration to 1 mg/mL. The measured absorbance of the obtained suspension at a wavelength of 570 nm was 0.3.

Subsequently, polyethylene glycol (molecular weight: 40,000) was biotinylated by a known method to prepare a biotinylated polyethylene glycol as an aggregation inhibitor. The biotinylated polyethylene glycol was added to the above-prepared suspension of magnetic particles so as to reach a concentration of 2.5 ng/mg. The measured absorbance of the magnetic particle suspension after the addition of the biotinylated polyethylene glycol at a wavelength of 570 nm was 0.15. The increased absorbance is an indicator of particle aggregation. Accordingly, it was successfully confirmed that the aggregation of magnetic particles was inhibited by adding the biotinylated polyethylene glycol as an aggregation inhibitor. More specifically, the biotinylated polyethylene glycol as an aggregation inhibitor bound to the magnetic particles with avidin as a target substance, thereby allowing the aggregation to be inhibited.

### Example 1

The photosensitizer-loaded particles prepared above and the fluorescent dye-loaded particles prepared above were suspended in water so as to be each 10 pM in particle number concentration. Polyallylamine as an aggregation enhancer was added to this suspension to reach a concentration of 0.01% by mass, and mixed at 25° C. In this state, each particle is stably dispersed.

The obtained dispersion was transferred to a quartz cuvette, and the dispersion in the cuvette was first irradiated with a semiconductor laser (output: 7.5 mW) with a wavelength of 685 nm as the first excitation light for exciting the sensitizer. Subsequently, the dispersion liquid in the cuvette was irradiated with a semiconductor laser (output: 1.0 mW) with a wavelength of 405 nm as a second excitation light for exciting the fluorescent dye. Thereafter, the fluorescence emitted from the dispersion was detected with a photomultiplier to obtain an output of 10 (arbitrary unit) after 1 second. It is to be noted that the increased output value represents stronger fluorescence.

Subsequently, the temperature of the dispersion in the cuvette was increased up to 43° C., and then irradiated with the first excitation light and the second excitation light in the same manner as mentioned above. Thereafter, the fluorescence emitted from the dispersion was detected with a photomultiplier to obtain an output of 1.5 (arbitrary unit).

As described above, the aggregation of the 10 pM photosensitizer-loaded particles and fluorescent dye-loaded particles with the temperature as a stimulus has been successfully confirmed by the fluorescence detection.

### Example 2

The carboxylic acid of the polymer compound mainly constituting the fluorescent dye-loaded particles mentioned above was subjected to biotinylation to obtain fluorescent dye-loaded particles with a biotin-derived structure as a target binding site. An evaluation was made with the obtained biotinylated fluorescent dye-loaded particles as a “substance that captures the target substance” and streptavidin as the “target substance”.

A solution was prepared in which the obtained fluorescent dye-loaded particles, biotinylated polyethylene glycol (molecular weight: 40,000), and the above-mentioned photosensitizer-loaded particles were suspended or dissolved in water so as to respectively have concentrations of 10 pM, 20 pM, and 10 pM. Furthermore, polyallylamine as an aggregation enhancer was added to the obtained solution so as to reach a concentration of 0.01% by mass, and mixed at 25° C.

The solution in this state was irradiated with the first excitation light and the second excitation light in the same manner as in Example 1, and then, the fluorescence emitted from the solution was detected with a photomultiplier to obtain an output of 10 (arbitrary unit) after 1 second.

Subsequently, the temperature of the solution was increased up to 43° C., the solution was irradiated with the first excitation light and the second excitation light in the same manner as in Example 1, and the fluorescence emitted from the solution was detected with a photomultiplier to obtain an output of 1 (arbitrary unit) after 1 second.

Newly, in the same manner as mentioned above, a solution was prepared in which the fluorescent dye-loaded particles with a biotin-derived structure, the biotinylated polyethylene glycol (molecular weight: 40,000), and the photosensitizer-loaded particles were suspended or dissolved in water to have respective concentrations of 10 pM, 20 pM, and 10 pM. Furthermore, polyallylamine as an aggregation enhancer was added to the obtained solution so as to reach a concentration of 0.01% by mass, and mixed at 25° C.

Thereafter, streptavidin as a target substance was further mixed with the obtained solution so as to reach a concentration of 5 pM, and then, the temperature of the solution was increased up to 43° C. Subsequently, the solution was irradiated with the first excitation light and the second excitation light in the same manner as in Example 1, and the fluorescence emitted from the solution was detected with a photomultiplier to obtain an output of 7 (arbitrary unit) after 1 second.

As described above, the clear difference in fluorescence output obtained has been successfully confirmed between the case of the sample containing no streptavidin as a target substance and the case of the sample containing streptavidin to have a concentration of 5 pM, and the 5 pM target substance (streptavidin) has been successfully detected from the fluorescence. This indicates that the structure derived from the biotin contained in the aggregation inhibitor and the fluorescent dye-loaded particles binds to streptavidin as a target substance, thereby allowing the aggregation of the fluorescent dye-loaded particles and photosensitizer-loaded particles to be inhibited.

As mentioned above, according to the present invention, a diagnostic agent, diagnostic kit, and diagnostic method are provided, which enable quick, inexpensive, simple, highly sensitive, and highly accurate detection or quantification of a target sub stance.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2020-033560, filed Feb. 28, 2020, which is hereby incorporated by reference herein in its entirety.

What is claimed is:

1. A diagnostic agent for use in detecting a target substance in a specimen, the diagnostic agent comprising a mixture of:
a photosensitizer-loaded particle containing a photosensitizer that generates singlet oxygen during light irradiation;
a fluorescent dye-loaded particle containing a fluorescent dye that changes in luminescent characteristics depending on an oxidation reaction with the singlet oxygen; and
an aggregation inhibitor that inhibits aggregation of the photosensitizer-loaded particle and the fluorescent dye-loaded particle,
wherein at least any one of the photosensitizer-loaded particle and the fluorescent dye-loaded particle has a property of binding to the target substance, and
a surface of the photosensitizer-loaded particles and a surface of the fluorescent dye-loaded particles have a stimulus-responsive polymer.

2. The diagnostic agent according to claim 1, wherein the aggregation inhibitor has a property of binding to the target substance.

3. The diagnostic agent according to claim 1, wherein the stimulus-responsive polymer is a temperature-responsive polymer.

4. The diagnostic agent according to claim 1,
wherein the photosensitizer is at least one selected from the group consisting of a porphyrin that may have a substituent, a phthalocyanine that may have a substituent, a fluorescein that may have a substituent, and a methylene blue, the porphyrin and the phthalocyanine may each have a central metal,
the substituents that may be included in the porphyrin, the phthalocyanine, and the fluorescein are each a halogen atom, a linear or branched alkyl group, an alkoxy group, an aryl group that may have a substituent, or a heterocyclic group that may have a substituent,
the substituent that may be included in the aryl group is a halogen atom, a linear or branched alkyl group, an alkoxy group, an aryl group, or a heterocyclic group,
the substituent that may be included in the heterocyclic group is a halogen atom, a linear or branched alkyl group, an alkoxy group, or an aryl group, and
the heterocyclic groups that may be included in the porphyrin, the phthalocyanine, and the fluorescein, and the heterocyclic group that may be included in the aryl group are each independently any group selected from the group consisting of a pyridyl group, an oxazolyl group, an oxadiazolyl group, a thiazolyl group, a thiadiazolyl group, a carbazolyl group, an acridinyl group, and a phenanthrolyl group.

5. The diagnostic agent according to claim 1,
wherein the fluorescent dye is an anthracene that may have a substituent, or benzofuran that may have a substituent,
the substituents that may be included in the anthracene and the benzofuran are each a halogen atom, a linear or branched alkyl group, an alkoxy group, an aryl group that may have a substituent, or a heterocyclic group that may have a substituent,
the substituent that may be included in the aryl group is a halogen atom, a linear or branched alkyl group, an alkoxy group, an aryl group, or a heterocyclic group,
the substituent that may be included in the heterocyclic group is a halogen atom, a linear or branched alkyl group, an alkoxy group, or an aryl group, and
the heterocyclic groups that may be included in the anthracene and the benzofuran, and the heterocyclic group that may be included in the aryl group are each independently any group selected from the group consisting of a pyridyl group, an oxazolyl group, an oxadiazolyl group, a thiazolyl group, a thiadiazolyl group, a carbazolyl group, an acridinyl group, and a phenanthrolyl group.

6. The diagnostic agent according to claim 1, wherein the stimulus-responsive polymer is at least any one selected from the group consisting of poly(N-alkylacrylamide), poly(N-vinylalkylamide), and polyvinyl alkyl ether.

7. The diagnostic agent according to claim 1, wherein the stimulus-responsive polymer is poly(N-isopropylacrylamide).

8. The diagnostic agent according to claim 1,
wherein the aggregation inhibitor comprises a hydrophilic site, and
the hydrophilic site has a structure represented by removing one hydrogen atom from at least any one selected from the group consisting of polyethylene glycol, polyvinylpyrrolidone, polyvinyl alcohol, sodium polyacrylate, polyethyleneimine, and copolymers thereof except for one hydrogen atom.

9. A diagnostic kit for use in producing the diagnostic agent according to claim 1, the diagnostic kit comprising the photosensitizer-loaded particle, the fluorescent dye-loaded particle, and the aggregation inhibitor,
wherein at least any one of the photosensitizer-loaded particle, the fluorescent dye-loaded particle, and the agglutination inhibitor is stored separately.

10. The diagnostic kit according to claim 9, wherein the photosensitizer-loaded particle, the fluorescent dye-loaded particle, and the aggregation inhibitor are all stored separately from each other.

11. A diagnostic agent for use in detecting a target substance in a specimen, the diagnostic agent comprising:
a photosensitizer-loaded particle containing a photosensitizer that generates singlet oxygen by light irradiation;
a fluorescent dye-loaded particle containing a fluorescent dye that changes in luminescent characteristics depending on an oxidation reaction with the singlet oxygen;
an aggregation inhibitor that has a property of inhibiting aggregation of the photosensitizer-loaded particle and the fluorescent dye-loaded particle by binding to the target substance; and
an aggregation enhancer that accelerates aggregation of the photosensitizer-loaded particle and the fluorescent dye-loaded particle,
wherein at least any one of the photosensitizer-loaded particle and the fluorescent dye-loaded particle has a property of binding to the target substance.

12. A diagnostic kit for use in producing the diagnostic agent according to claim 11, the diagnostic kit comprising the photosensitizer-loaded particle, the fluorescent dye-loaded particle, and the aggregation inhibitor,
wherein at least any one of the photosensitizer-loaded particle, the fluorescent dye-loaded particle, and the agglutination inhibitor is stored separately.

13. The diagnostic kit according to claim 12, wherein the photosensitizer-loaded particle, the fluorescent dye-loaded particle, and the aggregation inhibitor are all stored separately from each other.

14. A diagnostic method for detecting a target substance in a specimen, the diagnostic method comprising:
obtaining a test mixture by mixing the specimen, a photosensitizer-loaded particle containing a photosensitizer that generates singlet oxygen during light irradiation, a fluorescent dye-loaded particle containing a fluorescent dye that changes in luminescent characteristics depending on an oxidation reaction with the singlet oxygen, and an aggregation inhibitor that inhibits aggregation of the photosensitizer-loaded particle and the fluorescent dye-loaded particle, wherein at least any one of the photosensitizer-loaded particle and the fluorescent dye-loaded particle has a property of binding to the target substance, and a surface of the photosensitizer-loaded particles and a surface of the fluorescent dye-loaded particles have a stimulus-responsive polymer;
changing an environmental condition for the test mixture to change a property of the stimulus-responsive polymer;
irradiating the test mixture with first excitation light; and
irradiating the test mixture with second excitation light and detecting light emitted from the test mixture.

15. The diagnostic method according to claim 14, wherein the stimulus-responsive polymer is a temperature-responsive polymer.
